(19) United States
(12) Patent Application Publication
(10) Pub. No.: US 2025/0366909 A1
(43) Pub. Date: Dec. 4, 2025

(54) SYSTEMS AND METHODS FOR ENDOVASCULAR TREATMENT OF A BLOOD VESSEL

(71) Applicant: TVA Medical, Inc., Franklin Lakes, NJ (US)

(72) Inventors: Oladipo Peter Akerele-ale, Franklin Lakes, NJ (US); Andy Moll, Franklin Lakes, NJ (US); Alex Palmer, Franklin Lakes, NJ (US); Olivia Palmer, Franklin Lakes, NJ (US); Kristin Rominger, Franklin Lakes, NJ (US); Breanna Simpson, Franklin Lakes, NJ (US)

(21) Appl. No.: 18/873,552

(22) PCT Filed: Jun. 10, 2022
(86) PCT No.: PCT/US2022/032943

§ 371 Date: Dec. 10, 2024

(51) Int. Cl. A61B 18/14; A61B 18/00

(52) U.S. Cl. CPC A61B 18/1492; A61B 2018/00404; A61B 2018/00601; A61B 2018/00875

(57) ABSTRACT

A system for endovascular treatment of a blood vessel is provided. The system includes a first catheter having a housing and an electrode coupled to the housing, a second catheter having a backstop, the backstop comprising a cut completion sensor configured to output a signal indicative of the electrode completing a cutting operation from a first vessel to a second vessel, and a controller communicatively coupled to the cut completion sensor. The controller is operable to receive the signal from the cut completion sensor, and determine a status of the cutting operation based on the signal from the cut completion sensor.

### TECHNICAL FIELD

The present disclosure generally relates to systems and methods for treatment of a blood vessel, and more specifically, systems and methods for forming a fistula or providing other endovascular treatment.

### BACKGROUND

Endovascular treatments treat various blood vessel disorders from within the blood vessel using long, thin tubes called catheters, which are place inside the blood vessel to deliver the treatment. Endovascular treatments may include, but are not limited to, endovascular arteriovenous fistula (endoAVF) formations, arteriovenous (AV) treatments, and peripheral arterial disease (PAD) treatments.

One of challenging aspects of endovascular treatment is proper determination of completion of cutting operation of catheters. Treatments such as endovascular fistula formation may require two catheters positioned within adjacent blood vessels and cut the blood vessels to form a fistula therebetween. To determine completion of cutting operation of catheters, practitioners are trained to observe the electrode movement during activation of the device under active fluoroscopy, which can be difficult for practitioners. The visual determination by practitioners is also subjective. Additionally, practitioners may, in some cases, use cameras or similar devices to observe the flow path of fluid through the fistula. However, such processes require the catheters to be removed from the subject. Where the procedure was unsuccessful, reinsertion of the catheters may not be possible, leading to using additional catheter systems.

Accordingly, a need exists for systems and methods for endovascular treatment of a blood vessel that improve determination of cutting operation of catheters for formation of fistula or other endovascular treatment of a blood vessel that allow simpler determination of treatment of the blood vessel.

### SUMMARY

As noted above, one of challenging aspects of endovascular treatment is proper determination of completion of cutting operation of catheters. Embodiments of the present disclosure directed to systems and methods for endovascular treatment that provide improved determination of treatment of blood vessels will be described in greater detail below.

In one embodiment, a system for endovascular treatment of a blood vessel is provided. The system may include a first catheter having a housing and an electrode coupled to the housing, a second catheter having a backstop. The backstop may include a cut completion sensor configured to output a signal indicative of the electrode completing a cutting operation from a first vessel to a second vessel. The system may include a controller communicatively coupled to the cut completion sensor. The controller may be operable to receive the signal from the cut completion sensor, and determine a status of the cutting operation based on the signal from the cut completion sensor.

In another embodiment, a system for endovascular treatment of a blood vessel is provided. The system may include a first catheter having a housing and an electrode coupled to the housing, a second catheter having a backstop, a cut completion sensor configured to output a signal indicative of the electrode completing a cutting operation from a first vessel to a second vessel, and a controller communicatively coupled to the cut completion sensor. The controller may be operable to receive the signal from the cut completion sensor and determine a status of the cutting operation based on the signal from the cut completion sensor.

In yet another embodiment, a method for endovascular treatment of a blood vessel is provided. The method may include advancing a first catheter within a first blood vessel, wherein the first catheter has a housing and an electrode coupled to the housing, advancing a second catheter within a second blood vessel, wherein the second catheter has a backstop, performing a cutting operation between the first blood vessel and the second blood vessel with the first catheter and the second catheter, receiving, with a controller, a signal from a cut completion sensor communicatively coupled to the controller, and determining, with a controller, a status of the cutting operation based on the signal received from the cut completion sensor.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a system of catheters for endovascular treatment of a blood vessel with an enlarged view of ends of the catheters, according to one or more embodiments shown and described herein;

FIG. 2 schematically depicts an example of an electrical system for use with the system of FIG. 1, according to one or more embodiments shown and described herein;

FIG. 3 schematically depicts another example of an electrical system for use with the system of FIG. 1, according to one or more embodiments shown and described herein;

FIG. 4 depicts a graph illustrating impedance measurement of the electrical system of FIG. 2 in use, according to one or more embodiments shown and described herein;

FIG. 5 schematically depicts an example of a backstop of a first catheter for the system of FIG. 1, according to one or more embodiments shown and described herein;

FIG. 6 schematically depicts another example of a backstop of a first catheter for the system of FIG. 1, according to one or more embodiments shown and described herein; and

FIG. 7 depicts a flowchart illustrating a method for endovascular treatment of a blood vessel with the system of FIG. 1, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to systems and methods for endovascular treatment of a blood vessel such as, but not limited to forming a fistula, wire crossing procedures, bypass procedures, etc. For example, a catheter may be placed in each of two adjacent blood vessels to cut blood vessels to form a fistula therebetween with the catheters. However, proper determination of completion of cutting operation of catheters may be difficult for practitioners. For example, substantial training and practice may be needed to properly observe catheters during a cutting operation under active fluoroscopy to identify whether or not a cut has been successfully made. Embodiments of the present disclosure provide improved determination of cutting operation of catheters for formation of fistula or other endovascular treatment of a blood vessel. For example, in some embodiments, a system for endovascular treatment of a blood vessel according to the present disclosure includes a first catheter having a first housing and an electrode coupled to the first housing and a second catheter having a second housing and a backstop coupled to the second housing. A cut completion sensor is configured to output a signal indicative of the electrode completing a cutting operation from a first vessel to a second vessel. A controller is communicatively coupled to the cut completion sensor. The controller is operable to receive the signal from the cut completion sensor, and determine a status of the cutting operation based on the signal from the cut completion sensor. Accordingly, a user may determine whether an operation has been successfully completed in real-time without need for removing the catheters. These and additional features and benefits will be described in greater detail herein.

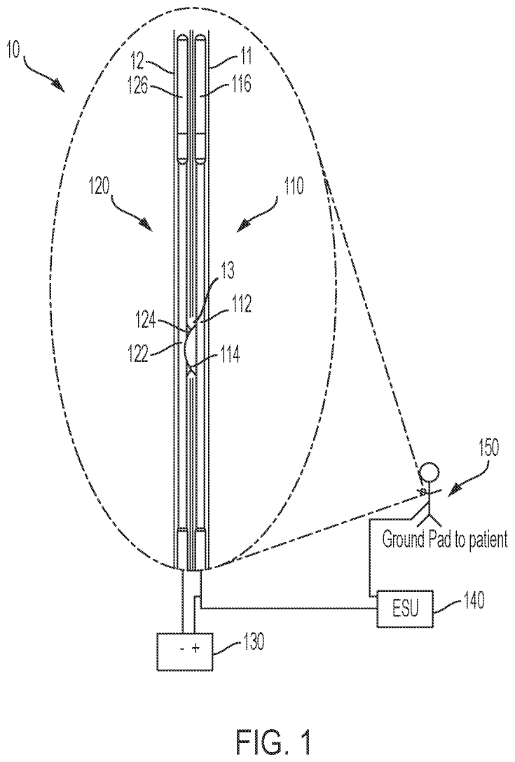

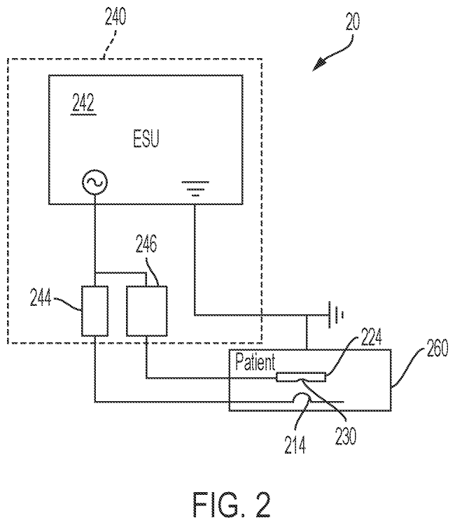

Referring now to FIG. 1, a catheter system 10 for providing endovascular treatment of a blood vessel, such as fistula formation, is schematically depicted. The catheter system 10 may include a first catheter 110 and a second catheter 120 to be inserted into a first blood vessel 11 and a second blood vessel 12, respectively, of a subject 150. For example, the first catheter 110 and the second catheter 120 may be inserted into adjacent blood vessels (e.g., an artery and a vein, a vein and a vein, an artery and an artery, etc.) in an arm (or other region) of the subject 150. The first catheter 110 and/or the second catheter may include a cut completion sensor 130 communicatively coupled to a controller 140. In some embodiments, the catheter system 10 may further include a ground pad (e.g., ground pad 260 shown in FIG. 2), which may be attached to a body of the subject 150 (e.g., via adhesive). In such embodiments, the ground pad may be communicatively coupled to the controller 140.

General deliverability features of the first catheter 110 and the second catheter 120 may be substantially similar to one another. Accordingly, features of the first catheter 110 may generally apply to the second catheter 120 unless otherwise noted or apparent. It is noted that the first catheter 110 and the second catheter 120 may be provided within a kit and/or separately from one another.

The first catheter 110 may be sized to be advanced through a blood vessel and may include a first distal tip 116, an electrode housing 112, and an electrode 114. It is noted that a greater or fewer number of components may be included as part of the first catheter 110 without departing from the scope of the present disclosure.

The first distal tip 116 may provide a distal end of the first catheter 110 that may be shaped and/or sized to aid in advancement of the first catheter 110 through a blood vessel. For example, the first distal tip 116 may be pointed, tapered, and/or atraumatic for advancement through a blood vessel. The first catheter 110 may have any cross-sectional shape and any diameter suitable for intravascular use (e.g., round, square, hexagonal, octagonal, etc.). The first catheter 110 may include or define one or more lumens or other passageways (not shown) extending at least partially along or through the first catheter 110. For instance, the one or more lumens may extend at least partially longitudinally through the catheter. The first catheter 110 may be formed of any material or combination of materials able to be traversed through a vasculature of a body. For example, the materials may include, silicone, rubber, etc.

In some embodiments, the electrode 114 disposed in the electrode housing 112 may be a wire, a spring wire, or a leaf spring, having an exposed ablation surface. The electrode 114 may be coupled to a power source (not shown) coupled to the controller 140, such as via a lead wire or other conductor attached thereto. The power source may be a radiofrequency current generator. When activated, current may be supplied to and/or carried from tissue and fluid via the ablation surface to facilitate ablation or vaporization of tissue to cut tissue and thereby to form a fistula.

In some embodiments, the first catheter 110 may comprise one or more insulating materials (not shown) which may shield or otherwise protect the first catheter 110 and its components from heat generated by the electrode 114 during use. For example, one or more portions of the electrode housing 112 may have one or more heat insulating portions which may include ceramic.

The size and shape of the electrode 114 be varied based on factors including tissue thickness and density, as well as desired fistula size, shape, and location. The electrode 114 may be arc shaped, though other shapes are contemplated and possible (e.g., rectangular, square, angular, etc.). The size and shape of the electrode 114 is not limited to as describe above, but may include a different cutting/ablation device such as, but not limited to, any electrocautery mechanism, blades, lances, needles, cryogenic-cautery devices, ultrasonic-cautery devices, laser ablation devices, etc.

Still referring to FIG. 1, the second catheter 120 may be sized to be advanced through a blood vessel and may include a second distal tip 126 and a backstop 124. In some embodiments, the backstop 124 may comprise the cut completion sensor 130 mounted on the backstop 124 instead of being coupled to the cut completion sensor 130. It is noted that the second catheter 120 may include a greater or fewer number of components without departing from the scope of the present disclosure.

The second distal tip 126 may be shaped and/or sized to aid in advancement of the second catheter 120 through a blood vessel. For example, the second distal tip 126 may be pointed, tapered, and/or atraumatic for advancement through a blood vessel. The second catheter 120 may have any cross-sectional shape and any diameter suitable for intravascular use. The second catheter 120 may include or define one or more lumens or other passageways (not shown) extending at least partially along or through the second catheter 120. For instance, the one or more lumens may extend at least partially longitudinally through the catheter. The second catheter 120 may be formed of any material or combination of materials able to be traversed through a vasculature of a body. For example, the materials may include, silicone, rubber, etc.

FIG. 1 illustrates alignment of the first catheter 110 and the second catheter 120. The electrode 114 of the first catheter 110 and the backstop 124 of the second catheter 120 may face to each other to be aligned to form a fistula. For example, when the electrode 114 is activated, the first blood vessel 11 and the second blood vessel 12 may be cut to form an opening 13, and the electrode 114 may become closer to the backstop 124 of the second catheter through the opening 13. The electrode 114 may come in contact with the backstop 124 through the opening 13. For example, energy is delivered to the electrode 114 that acts as a cutting tool for tissue ablation. During activation of the electrode 114, a plasma layer is created on the surface of the electrode 114 facing a tissue of a blood vessel. The plasma layer is pushed against the tissue, ablating the tissue to create a fistula.

The cut completion sensor 130 may detect completion of a cutting operation or contact of the electrode 114 and the backstop 124 through electrical signals. For example, when the electrode 114 and the backstop 124 become closer to each other or in contact with each other, the cut completion sensor 130 may detect changes in electrical signals indicative of the electrode 114 completing a cutting operation from the first blood vessel 11 to the second blood vessel 12. The cut completion sensor 130 may detect certain electrical signals indicative of the electrode 114 completing a cutting operation, such as impedance. Impedance is the opposition to current due to the effects of resistance and reactance (for alternating current systems). For direct current systems, impedance and resistance are the same and defined as the voltage across an element divided by the current (R=V/I). For alternating current systems, impedance is still measured in ohms, but voltage (V) and current (I) are frequency-dependent. In this regard, the impedance value changes with changes in either one or both of the voltage and the current. The controller 140 looking for relatively abrupt changes in current, voltage and/or impedance can signal a cutting operation and/or a completion of a cutting operation. Any sensors that are suitable for providing an output signal indicative of current, voltage and/or impedance may be used.

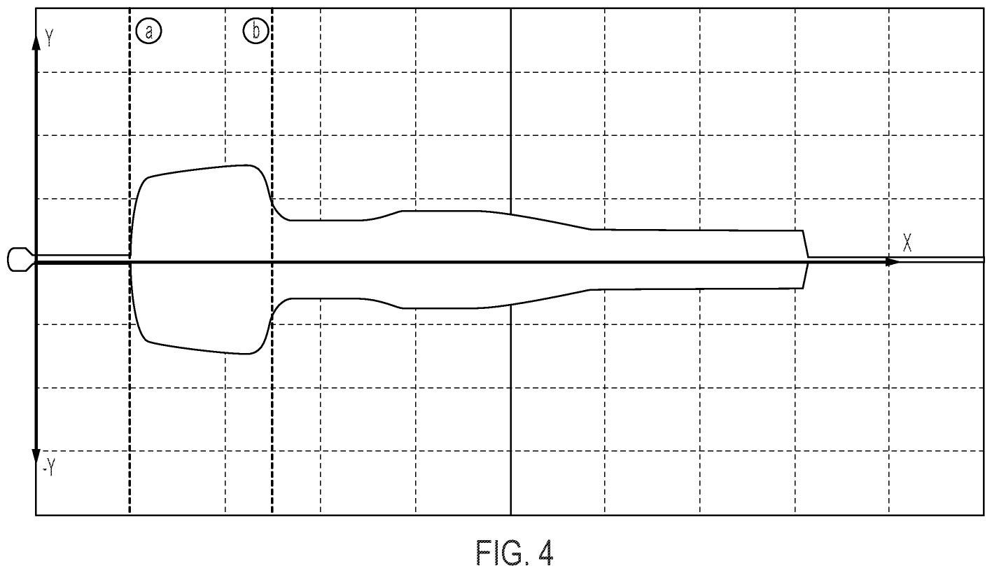

For example, the cut completion sensor 130 may be a probe embedded in the backstop 124. The probe may detect the impedance in the system during activation of the electrode 114 directly by measuring both the voltage and the current and providing an output indicative of impedance, and thereby used by the controller 140 using logic saved in memory to confirm interaction between the electrode 114 and the backstop 124. The cut completion sensor 130 may sense one or both of the current through the electrode 114 and the backstop 124, and the voltage across the electrode 114 and the backstop 124 and provide one or more signals to the controller 140 that can use the signals to determine the impedance. Multiple sensors can be used as the cut completion sensor 130 to provide outputs indicative of voltage, current and/or impedance. The output of the cut completion sensor 130 may be used to detect incompletion of a cutting operation or non-contact of the electrode 114 and the backstop 124 based on electrical signals or changes in electrical signals. The details of detection or measurement of electrical signals will be described in greater detail later in connection with FIG. 4.

Each of the first catheter 110 and the second catheter 120 may have alignment elements to assist alignment of the first catheter 110 and the second catheter 120. For example, alignment elements may be a magnetic array arranged on or within a catheter body of each of the first catheter 110 and the second catheter 120 and the magnetic array of the first catheter 110 and the magnetic array of the second catheter 120 may attract each other. The magnetic array may include a plurality of magnetic elements arranged in a longitudinal array along a length of the catheter. For example, the plurality of magnetic elements may be disposed along the catheter body of each of the first catheter 110 and the second catheter 120. It is noted that magnetic elements may be sized to be generally kept to the profile size of each of the first catheter 110 and the second catheter 120.

Generally, the dimensions of the magnets described herein may be selected based on the size of the catheters carrying the magnets, which in turn may be selected based on the anatomical dimensions of the blood vessels through which the catheters may be advanced. For example, if the catheter is to be advanced through a blood vessel having an internal diameter of about 3 mm, it may be desirable to configure any magnet to be less than about 3 mm at the widest part of its cross-section, to reduce the risk of injury to vessel walls during advancement and manipulation of the catheter. Each magnet may have any suitable length (e.g., about 5 mm, about 10 mm, about 15 mm, about 20 mm, or the like). In some embodiments, the number of the plurality of magnetic elements of the magnetic array may be modified for optimization of magnetic strength for alignment or coaptation purposes. The magnetic array may be continuous or may be broken in to a plurality of magnetic arrays, such as two or more magnetic arrays, three or more magnetic arrays, etc.

The magnetic elements may include permanent magnets comprising one or more hard magnetic materials, such as but not limited to alloys of rare earth elements (e.g., samarium-cobalt magnets or neodymium magnets, such as N52 magnets) or alnico. In some variations, the magnet elements may comprise anisotropic magnets; in other variations, the magnetic elements may comprise isotropic magnetics. In some variations, the magnetic elements may be formed from compressed powder. In some variations, the magnetic elements may include one or more soft magnetic materials, such as but not limited to iron, cobalt, nickel, or ferrite.

Referring to FIG. 2, in some embodiments, an electrical system 20 for use with the catheter system 10 of FIG. 1 may include a controller 240 communicatively coupled to an electrode 214 of a first catheter and a cut completion sensor 230 of a second catheter. The cut completion sensor 230 may be coupled to a backstop 224. For example, the cut completion sensor 230 may be incorporated in the backstop 224 of a first catheter (e.g., second catheter 120 of FIG. 1) such that the cut completion sensor 230 may be inserted into a first blood vessel of a subject. A ground pad 260 may be placed on a body of a subject. For example, the ground pad 260 may stick on the body of the subject. The subject may be grounded through the ground pad 260 coupled to a ground. The cut completion sensor 230 may be mounted to the backstop 224. For example, the cut completion sensor 230 may include a probe lead embedded in the backstop 224. The cut completion sensor 230 may be made with an electrically conductive material or any other suitable materials configured to sense a signal indicative of the electrode 214 completing a cutting operation. The cut completion sensor 230 may be communicatively coupled to the controller 240. The controller 240 may include an electrosurgical unit 242, a measurement device 246, and a user device 244. For example, the cut completion sensor 230 may be configured to output a signal detected from the cut completion sensor 230 to the measurement device 246. For example, the signal may be an impedance signal, radiofrequency signal, or a plasma generation signal. The radiofrequency signal may be a rate of oscillation of an electric current or voltage. The plasma generation signal may be a high frequency signal for establishing plasma. The measurement device 246 may provide the electrosurgical unit 242 with a measurement of the signal. The electrosurgical unit 242 may determine a status of the cut operation.

The measurement device 246 may measure a signal from the cut completion sensor 230. For example, the measurement device 246 may measure a signal from the cut completion sensor 230 to confirm energy delivery to the electrode 214, to confirm that a plasma phase (i.e., a highly energized state) is reached during a cutting operation, and/or to confirm that a cut is completed. Therefore, the controller 240 may receive the signal in the electrical system 20 and determine interaction between the electrode 214 and the cut completion sensor 230 and a status of cutting operation. The status of cutting operation may include activation of the electrode 214. In some embodiments, the measurement device 246 may be part of the second catheter with the cut completion sensor 230. The signal may also be used to determine reach of a plasma phase and/or completion of cutting.

The user device 244 may be multiple devices that are configured to receive input from a user. For example, the user device 244 may include a user interface such as a button or a touch screen. The electrosurgical unit 242 may activate the electrode 214 (i.e., generate and deliver energy to the electrode 214) to perform a cutting operation in response to an activation signal received from the user device 244. For example, a user may operate the user device to send an activation signal to the electrosurgical unit 242 to activate the electrode 214 to cut blood vessels and to form a fistula. The electrosurgical unit 242 may monitor the cutting operation with the cut completion sensor 230 throughout the cutting operation. For example, the electrosurgical unit 242 may determine a status of the cutting operation. In some embodiments, the user device 244 may include a device configured to create a light, an image, or a sound to communicate the status of the cutting operation to a user.

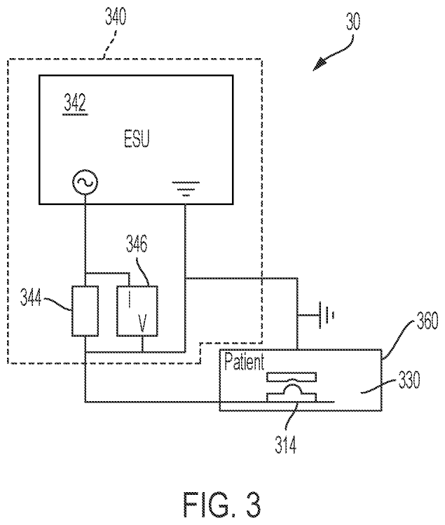

Referring to FIG. 3, in some embodiments, an electrical system 30 may include a controller 340 communicatively coupled to an electrode 314 of a first catheter and a ground pad 360 comprising a cut completion sensor 330 electrically connected to the ground pad 360. The ground pad 360 may be placed on a body of a subject. For example, the ground pad 360 may stick on to the body of the subject and coupled to the controller 340, and further coupled to a ground. The cut completion sensor 330 may be embedded in or otherwise be part of the ground pad 360. For example, the cut completion sensor 330 may be a probe lead embedded in the ground pad 360. The cut completion sensor 330 may be made with electrically conductive material, such as metal or any other suitable materials configured to sense a signal indicative of the electrode 314 completing a cutting operation. The cut completion sensor 330 may include, for nonlimiting example, an impedance meter, voltmeter, ammeter, or capacitive or resistive touch sensors. The cut completion sensor 330 may be communicatively coupled to a controller 340. The controller 340 may include an electrosurgical unit 342, a measurement device 346, and a user device 344. For example, the cut completion sensor 330 may be configured to output a signal detected from the cut completion sensor 330 to the measurement device 346. For example, the signal may be an impedance signal, radiofrequency signal, or a plasma generation signal. The measurement device 346 may provide the electrosurgical unit 342 with a measurement of the signal. The electrosurgical unit 342 may determine a status of the cut operation based on the signal measurement.

The measurement device 346 may measure a signal from the electrical system 30. For example, the measurement device 346 may measure a signal in the electrical system 30 to confirm energy delivery to the electrode 314, to confirm that a plasma phase (i.e., a highly energized state) is reached during a cutting operation, and/or to confirm that a cut is completed. Therefore, the controller 340 may receive the signal in the electrical system 30 and determine interaction between the electrode 314 and the cut completion sensor 330 to determine a status of cutting operation. For example, the impedance level from about 2 ohms to about 40 ohms may indicate that the plasma phase is reached during the cutting operation. In some embodiments, the measurement device 346 may be part of the ground pad 360 with the cut completion sensor 330. The signal may also be used to determine activation of the electrode 314 and/or reach of a plasma phase.

The user device 344 may be multiple devices that are configured to receive input from a user. For example, the user device 344 may include a user interface such as a button or a touch screen. The electrosurgical unit 342 may activate the electrode 314 (i.e., generate and deliver energy to the electrode 314) to perform a cutting operation in response to an activation signal received from the user device 344. For example, a user may operate the user device to send an activation signal to the electrosurgical unit 342 to activate the electrode 314 to cut blood vessels and to form a fistula. The electrosurgical unit 342 may monitor the cutting operation with the cut completion sensor 330 throughout the cutting operation. For example, the electrosurgical unit 342 may determine a status of the cutting operation. In some embodiments, the user device 344 may include a device configured to create a light, an image, or a sound to communicate the status of the cutting operation to a user.

A graph illustrated in FIG. 4 provides an example measurement of a signal from the system of any one of FIGS. 1 to 3. The graph plots measurement of a current signal from the system. An X-axis of the graph represents time, and an Y-axis of the graph represents current measurement. A vertical line-a represents activation of an electrode, and a vertical line-b represents completion of a cut. A plasma phase is reached between the line-a and the line-b. As shown in the graph, the current signal noticeably changes at a moment that the vertical line-b presents. This also indicates that impedance signal may noticeably change at the moment that the vertical line-b presents. For example, the changes in impedance may be about 500 ohms to about 2500 ohms.

The changes in the impedance signal may be used to determine the status of the plasma phase and/or the completion of a cut. For example, the completion of a cut may be determined based on a change itself in impedance measurement. Also, the completion of a cut may be based on a degree of change in impedance measurement. Further, the completion of a cut may be based on a value of impedance measurement. Threshold for determination of the completion of a cut may be set based on condition of the system, blood vessels, or other conditions that may affect the impedance level.

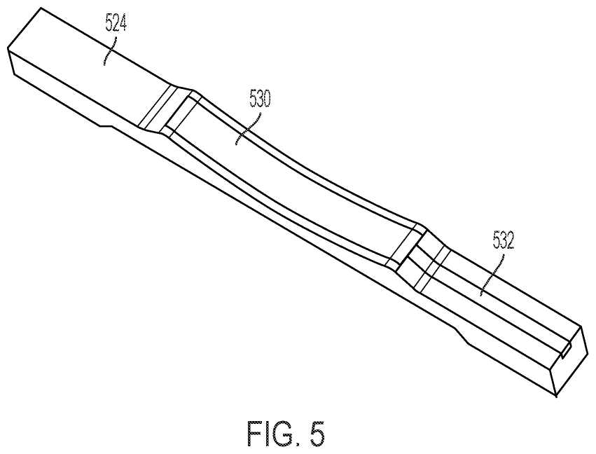

FIG. 5 illustrates some embodiments of a backstop having a cut completion sensor. A backstop 524 may have a cut completion sensor 530 mounted to the backstop 524. For example, the cut completion sensor 530 may be embedded in the backstop 524 such that the cut completion sensor 530 is disposed in the backstop 524 and the exposed surface of the cut completion sensor 530 is substantially flush with the exposed surface of the backstop 524. The cut completion sensor 530 may have a ribbon shape or a square shape. The cut completion sensor 530 may be communicatively coupled to a controller via a wire 532 (e.g., a lead wire or other conductor attached thereto) or wirelessly to transmit a signal. The signal may be an impedance signal, radiofrequency signal, or a plasma generation signal. The signal may be measured by a measurement device connected to the cut completion sensor 530.

The cut completion sensor 530 may be shaped to conform the exposed surface of the backstop 524 such that the cut completion sensor 530 may conform to an exposed surface of an electrode of a first catheter such that the cut completion sensor 530 and the electrode to contact each other when the electrode is activated to cut blood vessels disposed between the backstop 524 and the electrode to form a fistula. The cut completion sensor 530 may have a shape (e.g., a concave portion) that corresponds to and is complementary (e.g., inverse, reciprocal) to the electrode to match and conform to the electrode when the first catheter and the second catheter 520 are aligned and/or coapted. The shape of the cut completion sensor 530 may be varied based on factors including tissue thickness and density, as well as desired fistula size, shape, and location.

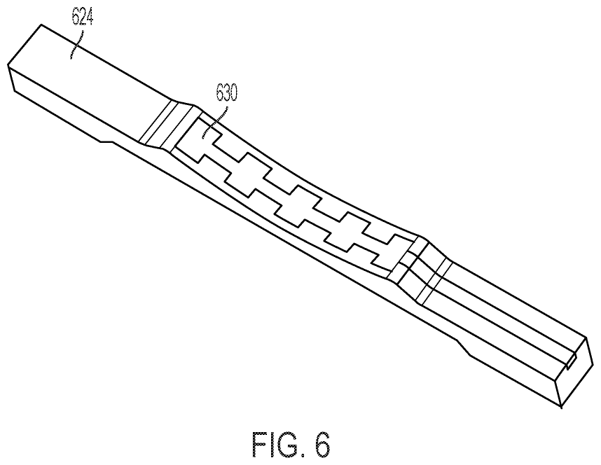

FIG. 6 illustrates other embodiments of a backstop having a cut completion sensor. A backstop 624 may have a cut completion sensor 630 mounted to the backstop 624. The cut completion sensor 630 is generally similar to the cut completion sensor 530 discussed above with respect to FIG. 5 except the shape. The cut completion sensor 630 may have toothed edges or castle line edges to improve sensing of a signal.

Configurations of a cut completion sensor are not limited to the above examples. In another embodiments, a cut completion sensor may have an array of sensor mounted to a backstop. For example, the array of sensor may be a group of sensors disposed along the longitudinal direction of the backstop. Individual sensors may provide individual signals to provide enhanced determination of a status of a cutting operation.

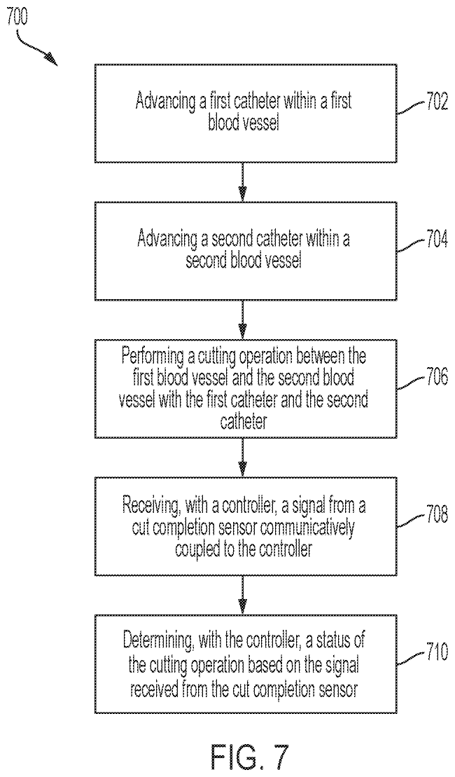

Referring to FIG. 7, a flow chart illustrating a method 700 of forming a fistula is generally depicted. It is noted that the method 700 may include a greater or fewer number of steps, taken in any order, without departing from the scope of the present disclosure. At block 702, the method 700 may include advancing a first catheter within a first blood vessel. The first catheter may have an electrode housing and an electrode coupled to the electrode housing. At block 704, the method 700 may include advancing a second catheter within a second blood vessel. The second catheter may have a backstop. It is noted that the first blood vessel and the second blood vessel may be adjacent vessels such as a vein and an artery, though vein to vein and artery to artery treatments are contemplated and possible. At block 706, the method 700 may include performing a cutting operation between the first blood vessel and the second blood vessel with the first catheter and the second catheter. At block 708, the method 700 may include receiving, with a controller, a signal from a cut completion sensor communicatively coupled to the controller. Further, the cut completion sensor may be coupled to the backstop. Additionally, the signal may comprise an impedance signal, a radiofrequency signal, or a plasma generation signal. At block 710, the method 700 may include determining, with a controller, a status of the cutting operation based on the signal received from the cut completion sensor.

In some embodiments, the method 700 may further include placing a ground pad in contact with a subject, wherein the ground pad comprises the cut completion sensor. For example, the ground pad may be placed on a body of a subject to be treated. The ground pad may have direct contact with the body of the subject so that the signal can be read through the cut completion sensor.

In other embodiments, the method 700 may further include outputting a signal with the controller indicative of the status of the cutting operation as complete or incomplete. The method 700 may further include outputting a signal with one or more user interface devices communicatively coupled to the controller the status of the cutting operation. For example, a user may confirm the completion of cut by viewing the result displayed on a user device.

In yet another embodiments, the method 700 may further include receiving an activation signal from one or more user input devices communicatively coupled to the controller. For example, a user may push a button on a user device to activate the electrode of the first catheter. The method 700 may further include operating the electrode to perform the cutting operation in response to the activation signal received from the one or more user input devices. For example, the activation signal from the user device may activate the electrode to perform the cutting operation. The method 700 may further include monitoring the cutting operation with the cut completion sensor throughout the cutting operation. For example, the cutting operation may be monitored by measuring the signal, for example, but not limited to an impedance signal, radiofrequency signal, or a plasma generation signal of the signal from the cut completion sensor.

Embodiments can be described with reference to the following numerical clause:

1. A system for endovascular treatment of a blood vessel, the system comprising: a first catheter having a housing and an electrode coupled to the housing; a second catheter having a backstop, the backstop comprising a cut completion sensor configured to output a signal indicative of the electrode completing a cutting operation from a first vessel to a second vessel; and a controller communicatively coupled to the cut completion sensor, wherein the controller is operable to: receive the signal from the cut completion sensor; and determine a status of the cutting operation based on the signal from the cut completion sensor.

2. The system of any preceding clause, wherein the signal comprises an impedance signal, a radiofrequency signal, or a plasma generation signal.

3. The system of any preceding clause, wherein the controller is further configured to output a signal indicative of the status of the cutting operation as complete or incomplete.

4. The system of any preceding clause, further comprising one or more user interface devices communicatively coupled to the controller, wherein the controller is further configured to output a signal indicative of the status of the cutting operation with the one or more user interface devices.

5. The system of any preceding clause, further comprising one or more user input devices communicatively coupled to the controller, wherein the controller is further configured to: receive an activation signal from the one or more user input devices; operate the electrode to perform the cutting operation in response to the activation signal received from the one or more user input devices; and monitor the cutting operation with the cut completion sensor throughout the cutting operation.

6. The system of any preceding clause, wherein the cut completion sensor comprises an array of sensors mounted to the backstop.

7. The system of any preceding clause, wherein the backstop is saddle-shaped and the cut completion sensor is mounted to the backstop within a concave portion of the backstop.

8. A system for endovascular treatment of a blood vessel, the system comprising: a first catheter having a housing and an electrode coupled to the housing; a second catheter having a backstop; a cut completion sensor configured to output a signal indicative of the electrode completing a cutting operation from a first vessel to a second vessel; and a controller communicatively coupled to the cut completion sensor, wherein the controller is operable to: receive the signal from the cut completion sensor; and determine a status of the cutting operation based on the signal from the cut completion sensor.

9. The system of clause 8, further comprising a ground pad configured to the placed in contact with a subject, the ground pad comprising the cut completion sensor.

10. The system of clauses 8 to 9, wherein the signal comprises an impedance signal, a radiofrequency signal, or a plasma generation signal.

11. The system of clauses 8 to 10, wherein the controller is further configured to output a signal indicative of the status of the cutting operation as complete or incomplete.

12. The system of clauses 8 to 11, further comprising one or more user interface devices communicatively coupled to the controller, wherein the controller is further configured to output a signal indicative of the status of the cutting operation with the one or more user interface devices.

13. The system of clauses 8 to 12, further comprising one or more user input devices communicatively coupled to the controller, wherein the controller is further configured to: receive an activation signal from the one or more user input devices; operate the electrode to perform the cutting operation in response to the activation signal received from the one or more user input devices; and monitor the cutting operation with the cut completion sensor throughout the cutting operation.

14. A method for endovascular treatment of a blood vessel, the method comprising: advancing a first catheter within a first blood vessel, wherein the first catheter has a housing and an electrode coupled to the housing; advancing a second catheter within a second blood vessel, wherein the second catheter has a backstop; performing a cutting operation between the first blood vessel and the second blood vessel with the first catheter and the second catheter; receiving, with a controller, a signal from a cut completion sensor communicatively coupled to the controller; and determining, with a controller, a status of the cutting operation based on the signal received from the cut completion sensor.

15. The method of any preceding clause, wherein the cut completion sensor is coupled to the backstop.

16. The method of any preceding clause, further comprising placing a ground pad in contact with a subject, wherein the ground pad comprises the cut completion sensor.

17. The method of any preceding clause, wherein the signal comprises an impedance signal, a radiofrequency signal, or a plasma generation signal.

18. The method of any preceding clause, further comprising outputting a signal with the controller indicative of the status of the cutting operation as complete or incomplete.

19. The method of any preceding clause, further comprising outputting a signal with one or more user interface devices communicatively coupled to the controller the status of the cutting operation.

20. The method of any preceding clause, further comprising: receiving an activation signal from one or more user input devices communicatively coupled to the controller; operating the electrode to perform the cutting operation in response to the activation signal received from the one or more user input devices; and monitoring the cutting operation with the cut completion sensor throughout the cutting operation.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system for endovascular treatment of a blood vessel, the system comprising:
a first catheter having a housing and an electrode coupled to the housing;
a second catheter having a backstop, the backstop comprising a cut completion sensor configured to output a signal indicative of the electrode completing a cutting operation from a first vessel to a second vessel; and
a controller communicatively coupled to the cut completion sensor, wherein the controller is operable to:
receive the signal from the cut completion sensor; and
determine a status of the cutting operation based on the signal from the cut completion sensor.

2. The system of claim 1, wherein the signal comprises an impedance signal, a radiofrequency signal, or a plasma generation signal.

3. The system of claim 1, wherein the controller is further configured to output a signal indicative of the status of the cutting operation as complete or incomplete.

4. The system of claim 1, further comprising one or more user interface devices communicatively coupled to the controller, wherein the controller is further configured to output a signal indicative of the status of the cutting operation with the one or more user interface devices.

5. The system of claim 1, further comprising one or more user input devices communicatively coupled to the controller, wherein the controller is further configured to:
receive an activation signal from the one or more user input devices;
operate the electrode to perform the cutting operation in response to the activation signal received from the one or more user input devices; and
monitor the cutting operation with the cut completion sensor throughout the cutting operation.

6. The system of claim 1, wherein the cut completion sensor comprises an array of sensors mounted to the backstop.

7. The system of claim 1, wherein the backstop is saddle-shaped and the cut completion sensor is mounted to the backstop within a concave portion of the backstop.

8. A system for endovascular treatment of a blood vessel, the system comprising:
a first catheter having a housing and an electrode coupled to the housing;
a second catheter having a backstop;
a cut completion sensor configured to output a signal indicative of the electrode completing a cutting operation from a first vessel to a second vessel; and
a controller communicatively coupled to the cut completion sensor, wherein the controller is operable to:
receive the signal from the cut completion sensor; and
determine a status of the cutting operation based on the signal from the cut completion sensor.

9. The system of claim 8, further comprising a ground pad configured to be placed in contact with a subject, the ground pad comprising the cut completion sensor.

10. The system of claim 8, wherein the signal comprises an impedance signal, a radiofrequency signal, or a plasma generation signal.

11. The system of claim 8, wherein the controller is further configured to output a signal indicative of the status of the cutting operation as complete or incomplete.

12. The system of claim 8, further comprising one or more user interface devices communicatively coupled to the controller, wherein the controller is further configured to output a signal indicative of the status of the cutting operation with the one or more user interface devices.

13. The system of claim 8, further comprising one or more user input devices communicatively coupled to the controller, wherein the controller is further configured to:
receive an activation signal from the one or more user input devices;
operate the electrode to perform the cutting operation in response to the activation signal received from the one or more user input devices; and
monitor the cutting operation with the cut completion sensor throughout the cutting operation.

14. A method for endovascular treatment of a blood vessel, the method comprising:
advancing a first catheter within a first blood vessel, wherein the first catheter has a housing and an electrode coupled to the housing;
advancing a second catheter within a second blood vessel, wherein the second catheter has a backstop;
performing a cutting operation between the first blood vessel and the second blood vessel with the first catheter and the second catheter;
receiving, with a controller, a signal from a cut completion sensor communicatively coupled to the controller; and
determining, with the controller, a status of the cutting operation based on the signal received from the cut completion sensor.

15. The method of claim 14, wherein the cut completion sensor is coupled to the backstop.

16. The method of claim 14, further comprising placing a ground pad in contact with a subject, wherein the ground pad comprises the cut completion sensor.

17. The method of claim 14, wherein the signal comprises an impedance signal, a radiofrequency signal, or a plasma generation signal.

18. The method of claim 14, further comprising outputting a signal with the controller indicative of the status of the cutting operation as complete or incomplete.

19. The method of claim 14, further comprising outputting a signal with one or more user interface devices communicatively coupled to the controller the status of the cutting operation.

20. The method of claim 14, further comprising:
receiving an activation signal from one or more user input devices communicatively coupled to the controller;
operating the electrode to perform the cutting operation in response to the activation signal received from the one or more user input devices; and
monitoring the cutting operation with the cut completion sensor throughout the cutting operation.
